(19) United States
(12) Patent Application Publication
(10) Pub. No.: US 2007/0275851 A1
(43) Pub. Date: Nov. 29, 2007

(54) Photocatalyst Particle Body

(76) Inventors: Tatsuo Sasano, Kawanishi-shi (JP); Yuichi Hashishin, Osaka-shi (JP); Masato Noda, Hashimoto-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/661,424

(22) PCT Filed: Aug. 18, 2005
(86) PCT No.: PCT/JP05/15075

§ 371 Date: Feb. 28, 2007

(30) Foreign Application Priority Data
Aug. 31, 2004 (JP) 2004-251480

(51) Int. Cl. B01J 35/02; A61L 9/00; B01J 35/08; C02F 1/32; C02F 1/72

(52) U.S. Cl. 502/159; 502/100; 502/208

(57) ABSTRACT

Photocatalyst particle bodies filled in optically transparent containers, which can fulfill the photocatalytic function without losing the function of the photocatalyst particles not only on the surface side but also on the inner side even when the insides of the containers are provided with a treatment structure that treated water passes through and are easily recovered and recycled are provided wherein photocatalyst fine particles of apatite-coated titanium dioxide or the like are contained in hollow shells made of a thermoplastic resin such as polystyrene having optical transparency, air permeability, and water permeability obtained by a drying in liquid method, and a particle body size thereof is not less than 1 millimeter.

### BACKGROUND OF THE INVENTION

1. Field of the Invention

The present invention relates to a photocatalyst particle body to be preferably used for purifying water and air.

2. Description of the Related Art

Some metal oxides have a photocatalytic function like titanium dioxide, and a method for deodorization, antibacterial treatment, removing hazardous gases, and purifying water, etc., using the decomposition action of powder particles thereof, that is, photocatalyst particles have already been used (for example, refer to Japanese Published Unexamined Patent Application No. 2003-260462).

The particle size of the photocatalyst particles is as small as 10 nanometers to 50 nanometers, and even in a state that the particles are aggregated, the particle size is not more than about 1 micrometer. Therefore, when water purification is performed by using the photocatalyst particles, a base material with a surface to which the photocatalyst particles are fixed by using a glue, etc., is installed within a water channel to purify water (sterilization, deodorization, etc.) passing through the inside of the water channel.

However, in the above-described method in which the photocatalyst particles are fixed to the base material surface, the base material shape must be changed each time the form and scale of the water purifying system change, and this increases the cost of equipment.

Therefore, the inventor of the captioned invention considered the possibility of solving the problem by making treated water to be purified pass through the inside of a cylindrical transparent container filled with photocatalyst particles. However, in this method, as described above, the photocatalyst particles were as fine as about 1 micrometer or less even in the state that they were aggregated, so that the photocatalyst particles were densely filled in the containers, and even if they were irradiated with light from the outside, the light reached only the photocatalyst particles on the surface side. Therefore, the photocatalyst particles on the lower layer and the inner sides could not fulfill the photocatalytic function, and the treated water could not smoothly flow.

In addition, it was found that the recovery and recycle of the photocatalyst particles were difficult. And as a result of further earnest study on this, the present invention was completed.

In view of these circumstances, an object of the present invention is to provide a photocatalyst particle body filled in optically transparent containers, which can fulfill the photocatalytic function and are easily recovered and recycled without losing the function of the photocatalyst particles not only on the surface side but also on the inner side even when the insides of the containers are provided with a treatment structure that treated water passes through.

### SUMMARY OF THE INVENTION

In order to achieve the object, a photocatalyst particle body of the present invention is formed of photocatalyst fine particles contained in a hollow shell with optical transparency, air permeability, and water permeability and the photocatalyst particle body has a particle size of not less than 1 millimeter.

In the present invention, the photocatalyst fine particles are not especially limited as long as they have a photocatalytic function, however, for example, there are available semiconductors of gallium phosphide (GaP), gallium arsenide (GaAs), cadmium sulfide (CdS), strontium titanate (SrTiO₃), titanium dioxide (TiO₂), zinc oxide (ZnO), ferricoxide (Fe₂O₃), tungsten oxide (WO₃), and the like. And when durability of the hollow shell is demanded, apatite-coated titanium dioxide (for example, refer to Japanese Published Unexamined Patent Application No. H10-244166) is preferably used.

In the present invention, apatite-coated titanium dioxide is not titanium dioxide completely coated with apatite (calcium phosphate), but is titanium dioxide a part of which is exposed to the outside.

A material of the hollow shell is not especially limited as long as it has optical transparency, air permeability, and water permeability, however, a thermoplastic resin is preferable in view of its excellent impact resistance and light weight.

The particle body size of the photocatalyst particle body is not especially limited, and preferably, not less than 2 millimeters and not more than 5 millimeters.

A method for producing the photocatalyst particle body is not especially limited. However, for example, when a hollow shell is made of a thermoplastic resin, there are available a drying in liquid method, a coacervation method, an aerial drying method, a method in which semispherical molded items are molded from a thermoplastic resin, the photocatalyst fine particles are filled in one of the obtained semispherical molded items, and then the other spherical molded item is closely fitted thereto while their open edges fit with each other, and this closely-fitted portion is bonded together by means of thermal fusion, and a method in which a tubular molded item is molded from a thermoplastic resin, and in a state that the photocatalyst fine particles are filled in the tubular molded item, the tubular molded item is thermally fused at predetermined pitches and the thermally fused portions are cut as appropriate, and in view of productivity and quality, a drying in liquid method is preferable. When an aerial, drying method is used, if a state of weightlessness is not realized, the hollow shells become distorted, so that a large-scale apparatus for creating a state of weightlessness is necessary to obtain spherical hollow shells, and this poses a problem with cost.

When a drying in liquid method is used, a solvent to be used is not especially limited, however, for example, dichloromethane, carbon tetrachloride, chloroform, etc., are available, and a material of a similar specific gravity close to that of the thermoplastic resin forming the hollow shell is preferably used.

The thermoplastic resin forming the shells is not especially limited, however, for example, a resin with high transparency such as polystyrene, polyvinyl chloride, low-density polyethylene, acryl resin, and acrylonitril-styrene copolymer, etc., are preferably used, and industrially, an inexpensive resin such as polystyrene, polyvinyl chloride, and low-density polyethylene, etc., are more preferably used. Still more preferably, a resin with low crystallinity is used.

Among these thermoplastic resins, a resin having a molecular weight of several hundred (the same level as an oligomer) and many lateral chains is preferable.

That is, when the molecular weight of the thermoplastic resin forming the shell is not so great and the resin has many lateral chains, molecules of the thermoplastic resin become suitably intertwined with each other and a hollow shell with an excellent network structure are formed and higher water permeability and air permeability can be secured.

The amount of the photocatalyst fine particles in the hollow shell is not especially limited, however, it is more preferable that the photocatalyst fine particles are sparsely filled to some degree so as to create gaps between the hollow shell and the photocatalyst fine particles than dense filling of the photocatalyst fine particles. That is, when gaps are left between the hollow shell and the photocatalyst fine particles, light that penetrated through the hollow shell and entered the inside of the hollow shell is reflected diffusely inside the hollow shell and easily irradiates the photocatalyst fine particles to the inner side.

The photocatalyst particle body of the present invention has a particle body size not less than 1 millimeter, so that even when the photocatalyst particle bodies are filled in optically-transparent containers and treated water passes through the insides of the containers, gaps which enable transmission of light from the outside to the photocatalyst particle bodies on the lower layer and inner sides are secured between the photocatalyst particle bodies. In addition, the hollow shell has optical transparency, air permeability, and water permeability, so that the photocatalyst fine particles inside the hollow shell are suitably irradiated with light from the outside through the hollow shell, and the photocatalyst fine particles can efficiently purify treated water that entered the hollow shell by means of the photocatalytic function thereof.

By using apatite-coated titanium dioxide as the photocatalyst fine particles, direct contact of titanium dioxide with the hollow shell is prevented by apatite, so that even when the hollow shell is made of a thermoplastic resin, etc., which is easily deteriorated by the photocatalytic function, the hollow shell can be prevented from being deteriorated by the photocatalytic function of titanium dioxide. Furthermore, apatite can keep bacteria near titanium dioxide, so that the sterilization effect increases further.

Furthermore, when the particle body size of the photocatalyst particle body is not less than 2 millimeters and not more than 5 millimeters, handling performance is excellent. In addition, when the photocatalyst particle bodies are filled in an optically-transparent container having water permeability and the container is placed into the treated water, the photocatalytic function can be effectively fulfilled due to the efficient light transmittance to the photocatalyst fine particles. If the particle body size of the photocatalyst particle body is more than 5 millimeters, photocatalyst fine particles at the central portion of the hollow shell are hardly irradiated with light and the photocatalytic efficiency may lower. When the photocatalyst particle body is produced by a drying in liquid method, if the particle body size of the photocatalyst particle body is more than 5 millimeters, the hollow shell becomes excessively thick and water and air permeability is deteriorated, and the photocatalyst efficiency may lower.

According to the drying in liquid method, the photocatalyst particle body can be efficiently produced at low cost, and the particle body size of the photocatalyst particle body obtained by controlling a stirring speed can be easily controlled. In addition, the hollow shell can be easily formed to be spherical.

### BRIEF DESCRIPTION OF THE DRAWING

FIG. 1 is a sectional view schematically showing an embodiment of photocatalyst particle body of the present invention.

### DETAILED DESCRIPTION OF THE PREFERRED EMBODIMENTS

Hereinafter, the present invention will be described in detail with reference to the drawing describing an embodiment.

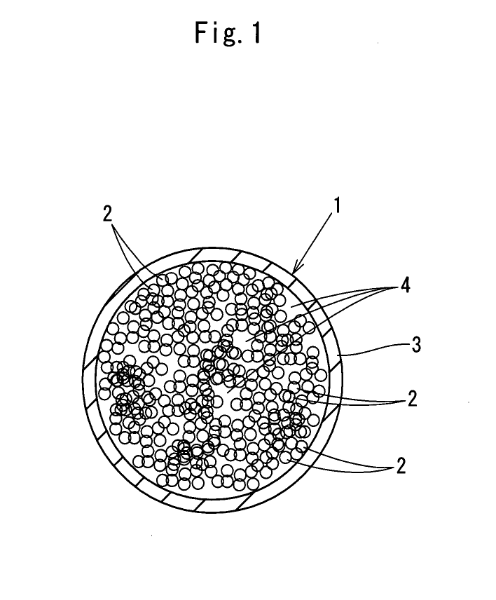

FIG. 1 schematically shows an embodiment of photocatalyst particle body of the present invention.

As shown in FIG. 1, the photocatalyst particle body 1 is formed by containing an aggregate of photocatalyst fine particles 2 of apatite-coated titanium dioxide or the like in a hollow shell 3 made of a thermoplastic resin with optical transparency, air permeability, and water permeability such as polystyrene into a particle body size of about 2 millimeters to 5 millimeters. The photocatalyst fine particles 2 and an aggregate thereof are not densely filled in the hollow shell 3, but are filled therein so that gaps 4 are left between the hollow shell 3 and the optical catalyst fine particles 2 and aggregate thereof.

This photocatalyst particle body 1 is filled in a bag or container with meshes slightly smaller than the particle body size of the photocatalyst particle body 1 and immersed in a treated water tank of a water purification plant or installed in the middle of a treated water flow channel, whereby treated water can be purified by the sterilization effect of the photocatalyst and the decomposition effect of organic matter due to oxidoreduction.

The photocatalyst particle body of the present invention can be produced by using, for example, the following drying in liquid method.

That is, according to this production method using the drying in liquid method, a resin solution in which a thermoplastic resin such as polystyrene with a melting point higher than a boiling point of an organic solvent such as dichloromethane is dissolved in the organic solvent is stirred while a slurry obtained by dispersing photocatalyst fine particles of apatite-coated titanium dioxide or the like is dripped therein to obtain a W/O (water-in-oil) dispersion in which the slurry is dispersed in a particle form in the resin solution, and then this W/O dispersion is further stirred while slowly being poured into another aqueous solution such as water, whereby a W/O/W dispersion is obtained.

Next, this W/O/W dispersion is heated to a temperature that is lower than the temperature of the thermoplastic resin and equal to or slightly higher than the boiling point of the organic solvent to evaporate the organic solvent, and the thermoplastic resin is solidified in water, where by hollow shells containing the slurry inside are formed. The hollow shells containing the slurry inside are taken out from the liquid, and dried and sieved as appropriate.

The evaporated dichloromethane can be recycled by recovering and aggregating it, so that the problem of environmental pollution can be solved.

### First Embodiment

200 grams of dichloromethane was put in a beaker, and while stirring dichloromethane by a stirring magnet by using a stirrer, 17.4 grams of polystyrene (pellet with 3 millimeter diameter×3 millimeter length, made by Kishida Chemical Co., Ltd.) was put into the beaker and dissolved in dichloromethane in about 1 hour, whereby a polystyrene solution with a concentration of 8 weight percent was obtained. Dichloromethane evaporates even at a room temperature, so that during dissolving, a polyvinylidene chloride film (registered trademark: Saran Wrap) was covered on the mouth of the beaker and the periphery thereof was stopped with a rubber band to prevent evaporation of dichloromethane.

While 30 ml of the apatite-coated titanium dioxide fine particle slurry (slurry in which apatite-coated titanium dioxide obtained by coating 20% of the surface of titanium dioxide ST21 made by Ishihara Sangyo Kaisha, Ltd. (average particle size: 20 nanometers) with apatite is dispersed at a proportion of 20 weight percent in water) was dripped in the polystyrene solution obtained as described above, stirring of the polystyrene solution was continued, whereby a W/O (water-in-oil) dispersion in which the slurry was dispersed in a particle form was obtained.

While the obtained dispersion was slowly poured into a beaker containing 800 ml of water, the dispersion was stirred for 1 hour by a stirring magnet by using a stirrer, whereby a W/O/W dispersion was obtained.

After leaving for a while, the W/O/W dispersion was slowly heated to 40 to 50 degrees Celsius that was substantially equal to or slightly higher than the boiling point of dichloromethane to evaporate dichloromethane in the dispersion while the temperature of 40 to 50 degrees Celsius was maintained. At this time, a part of polystyrene dissolved in dichloromethane formed a thin film on the surface of the dispersion, so that this film was removed well.

After dichloromethane was completely evaporated, heating was stopped and the dispersion was left and cooled while being stirred.

After leaving and cooling, the liquid in the beaker was filtrated with a filter paper, and shells remaining on the filter paper were dried, whereby the photocatalyst particle bodies were obtained.

The obtained photocatalyst particle bodies were sieved into particles with a diameter not more than 45 micrometers (hereinafter, referred to as “particles A”), particles with a diameter more than 45 micrometers and not more than 53 micrometers (hereinafter, referred to as “particles B”), particles with a diameter more than 53 micrometers and not more than 1.7 millimeters (hereinafter, referred to as “particles C”), and particles with a diameter more than 1.7 millimeters and not more than 2.0 millimeters (hereinafter, referred to as “particles D”).

The particles A through D obtained as described above and the apatite-coated titanium dioxide fine particles (hereinafter, referred to as “particles E”) were measured at 1 gram each and placed together with 19 grams of water in test tubes, respectively, and 3 drops of a methylene blue solution with a concentration of 250 ppm were dripped in each test tube, and then the test tubes were irradiated with an ultraviolet ray from a position of about 20 cm from the test tubes by using a 15 W black light with a peak wavelength of 352 nanometers for 150 minutes and colors of water in the test tubes were observed, and as a result, the discoloration degrees of methylene blue were D>C>B>A>E.

From these results, it is proved that the photocatalyst particle body of the present invention formed by containing apatite-coated titanium dioxide fine particles in a hollow shell has a function as a photocatalyst more excellent than in the case where the apatite-coated titanium dioxide fine particles are used in a state that the particles are not contained in shells.

The photocatalyst particle body of the present invention can be used for a water purifying system of a water purification plant, water purification of a water tank or bathtub, air purification (disinfecting and deodorization) by being incorporated in air conditioning equipment such as an air cleaner and air conditioner, keeping fresh agricultural products, a deodorizer in a bathroom or a room, and deodorization in a cattle house or shelter for farm animals or pet animals.

1. A photocatalyst particle body comprising:
A hollow shell with optical transparency, air permeability and water permeability; and
photocatalyst fine particles contained in the hollow shell,
wherein said photocatalyst particle body has a particle body size not less than 1 millimeter.

2. The photocatalyst particle body according to claim 1, wherein the photocatalyst fine particles are apatite-coated titanium dioxide.

3. The photocatalyst particle body according to claim 1, wherein the hollow shell is made of a thermoplastic resin.

4. The photocatalyst particle body according to claim 1, wherein the particle body size of the photocatalyst particle body is not less than 2 millimeters and not more than 5 millimeters.

5. The photocatalyst particle body according to claim 1, obtained by a drying in liquid method.

6. The photocatalyst particle body according to claim 2, wherein the hollow shell is made of a thermoplastic resin.

7. The photocatalyst particle body according to claim 2, wherein the particle body size of the photocatalyst particle body is not less than 2 millimeters and not more than 5 millimeters.

8. The photocatalyst particle body according to claim 3, wherein the particle body size of the photocatalyst particle body is not less than 2 millimeters and not more than 5 millimeters.

9. The photocatalyst particle body according to claim 2, obtained by a drying in liquid method.

10. The photocatalyst particle body according to claim 3, obtained by a drying in liquid method.

11. The photocatalyst particle body according to claim 4, obtained by a drying in liquid method.
